(19) United States
(12) Plant Patent Application Publication
(10) Pub. No.: US 2003/0028943 P1
(43) Pub. Date: Feb. 6, 2003

(54) Gladiolus plant named 'Prissy's Purple'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/918,662

(22) Filed: Jul. 31, 2001

(51) Int. Cl. A01H 5/00

(52) U.S. Cl. PLT/301

(57) ABSTRACT

A new and distinct gladiolus variety, designated ‘Prissy's Purple’, is shown and described. Compared to the ‘Spartan’ variety, the ‘Prissy's Purple’ variety has a stem length that is 10-15 cm longer, contains 2 to 4 more florets per flower head, and can hold open 3 to 4 more florets.

The present invention comprises a new and distinct variety of a Gladiolus plant referred to by the variety name ‘Prissy's Purple.’

### BRIEF DESCRIPTION OF THE PHOTOGRAPHS

FIG. 1 is a photograph of a ‘Prissy's Purple’ variety plant in bloom.

### ASEXUAL REPRODUCTION

The new variety was originated by the Applicant in a controlled proprietary breeding program in Ft. Myers, Fla. wherein selected gladiolus varieties were crossed (the Applicant's selected provisional breeder's designation is 26-2). The female parent was a rich purple gladiolus variety named ‘Spartan,’ an unpatented released variety characterized in part by having heavy, but not particularly tall stems (i.e. 115-120 cm from Jumbo size bulbs) and week resistance to Fusarium species. The male parent was a violet gladiolus variety named ‘Poet's Dream,’ a released variety characterized in part by having tall, heavy stems. The seeds were planted in Ft. Myers, Fla., and the selection of ‘Prissy's Purple’ variety was made in Spring 1988. Asexual reproduction of the ‘Prissy's Purple’ variety was achieved by collecting cormels from the first corm. All subsequent asexual reproductions of the ‘Prissy's Purple’ variety remain true to the original variety type.

### BOTANICAL DESCRIPTION

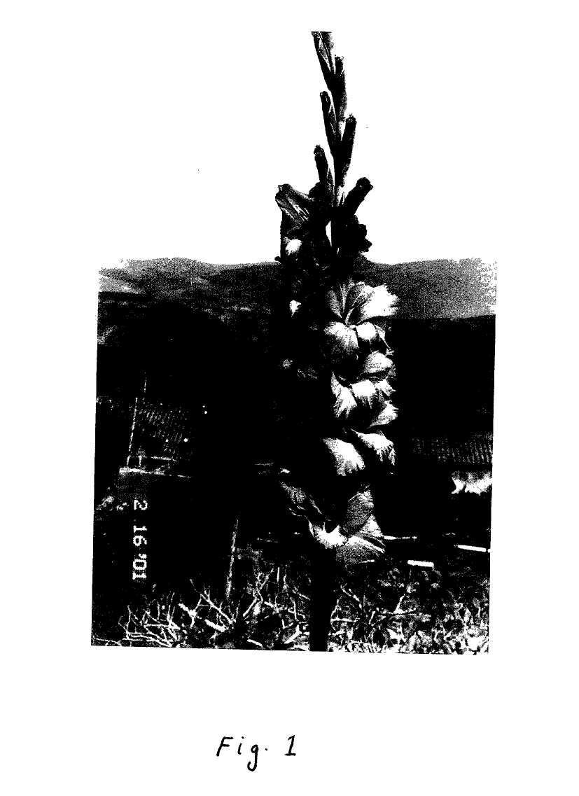

The accompanying color photograph (FIG. 1) shows the inflorescence and various stages of blooming of the ‘Prissy's Purple’ variety plant.

The bud size of the ‘Prissy's Purple’ variety is about 6.5 cm in length with a bloom diameter of 12 cm. The flowers of the variety each consist of a total of six petals, namely three large petals, two medium-size petals, and one small petal. Generally, the large petals are oriented on the top half of the flower, the two medium flowers are oriented on the lower half of the flower (about 4:00 and 8:00), and the small flower is positioned between the two medium petals (about 6:00).

The color of the corolla bloom is white with purple specks deep within the throat. The large petals are a uniform violet purple with darker purple flecks on the margin. The medium size petals are a uniform violet purple having a white line running longitudinally up the middle of the petal. The small petal is white on the inner part of the petal with a white line running up the center of the petal. The small petal is purple on the outer part of the petal.

The spike of the plant is about 128 cm and contains 20-22 florets when grown from Jumbo size corms (1.75 inch and larger) in Ecuador. The head length is about 68 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are dark purple.

The corms of the ‘Prissy's Purple’ variety are typical for the gladiolus.

The leaf color of the ‘Prissy's Purple’ variety is green. The leaf size is about 80 cm (length) by 4.8 cm (width).

### GENERAL OBSERVATION

Both the ‘Prissy's Purple’ variety and ‘Spartan’ variety are late to harvest in Ecuador, taking about 4-6 days longer than the Advance gladiolus variety. The ‘Prissy's Purple’ variety provides excellent growth in mid-winter, spring, and summer days, whereas the Spartan variety prefers the longer day lengths of the spring season. The ‘Prissy's Purple’ variety has good tolerance to heat.

The variety usually produces one to two stems per Jumbo size corm. The variety can maintain up to nine flowers in open bloom simultaneously in a vase of water and can be harvested with one flower showing color. The variety is an excellent opener after transport, in that the stems may be cut tight, shipped dry for a week, and still bloom well afterwards.

The ‘Prissy's Purple’ variety has good resistance to fungi diseases and is much more resistant to attack by Fusarium fingi than the ‘Spartan’ variety.

Compared to the ‘Spartan’ variety, the ‘Prissy's Purple’ variety contains 2 to 4 more florets per flower head, can hold open 3 to 4 more florets, and has a stem length that is 10-15 cm longer.

I claim:

1. A new and distinct gladiolus variety ‘Prissy's Purple’, as shown and described herein.
